(19) United States
(12) Patent Application Publication
(10) Pub. No.: US 2003/0036109 A1
(43) Pub. Date: Feb. 20, 2003

(54) Diagnostic analytical method and kit

(76) Inventors: Simak Pour Yazdankhah, As (NO); Geir Olav Gogstad, Oslo (NO); Rune Henning Sorum, Oslo (NO); Hans Jorgen Soiland Larsen, Oslo (NO)

Correspondence Address: Karen Lee Orzechowski, Liniak Berenato Longacre & White, Suite 240, 6550 Rock Spring Drive, Bethesda, MD, 20817, US

(21) Appl. No.: 10/169,256

(22) PCT Filed: Dec. 22, 2000
(86) PCT No.: PCT/GB00/04986

(30) Foreign Application Priority Data
Dec. 29, 1999 (NO) 19996532
Oct. 24, 2000 (NO) 20010822
Nov. 8, 2000 (NO) 20010823
Nov. 27, 2000 (NO) 20010824

(51) Int. Cl. C12Q 1/04; G01N 33/04

(52) U.S. Cl. 435/40.5; 436/23

(57) ABSTRACT

The invention provides a method for analyzing for bacteria in a liquid sample comprising the following steps: a) contacting said sample with a substrate capable of retaining bacteria or bacterial fragments; b)staining any bacteria retained on said substrate with at least one solution comprising a first dye, said staining being conducted at a pH at which the dye has an overall positive charge and the bacteria has an overall negative charge; and c) inspecting the substrate for the presence of a stain whereby to indicate the presence of bacteria in said sample; with the proviso that where said liquid sample is other than a milk sample and substrate is a filter then said filter comprises a polysulfone or a derivative of a polysulfone.

This invention relates to a kit and a method for detecting the presence of bacteria, and for classification of bacteria in samples with respect to sensitivity to various antibiotics. The method is applicable to classification of bacteria in any liquid sample, or in solid samples after liquid extraction. In a preferred version of the method, however, the sample is a human or animal body liquid such as blood, plasma, serum, mucous, semen, sputum, urine or milk and in a specially preferred version, the method may be used to detect the presence of, and to classify bacteria in milk from animals with mastitis.

Bacterial infections frequently cause human and animal diseases, with considerable economic and social implications. Since Fleming's discovery of penicillin in 1928, a variety of antibiotic agents have been developed for treatment of bacterial infections.

The application of antibiotics is, however, not without adverse environmental side effects. The widespread use of antibiotics has resulted in a gradual development of resistant bacteria. This phenomenon is particularly prevalent in environments where there is frequent use of antibiotics, for example in hospitals. Such environments experience ever-increasing instances of infections which necessitate alternative treatment with more broadly acting antibiotics. The consequence is an exacerbation of the problem of resistance and thus a concomitant reduction in the therapeutic potential of antibiotics.

This trend is a global problem which eventually may lead to loss of efficiency and even loss of efficacy of one of our most prized therapeutic tools. The long term consequence to human and animal health may be severe. The problem is augmented by the widespread prophylactic use of antibiotics in agriculture and aquaculture. Trace amounts of antibiotics are transferred to the consumers of fish, meat and milk, increasing the risk of spreading the antibiotic resistance from animals to humans. There has also been recorded an increased frequency of allergic reactions to antibiotics. This is explained by the spill-over into nutrients. In order to stop or slow, or even to reverse this trend, the World Health Organisation (WHO) has initiated an action plan which is directed towards reducing globally the quantity of antibiotics used.

Analytical methods which can be used to determine whether an infection is caused by bacteria, and if that is the case, to verify which class or classes of bacteria is involved, will be important tools in enabling targeting of the use of antibiotics. Such targeting will play a pivotal role in engineering a reduction in the application of broad-spectrum antibiotics to instances where their use is absolutely necessary.

One of the greatest economic losses suffered by cattle producers and milk farmers is caused by the disease mastitis. The Veterinary Institute and Norske Meierier estimates that, in Norway, the direct and indirect annual loss caused by this disease is about 300 million Norwegian Kroner. The annual loss on a European basis is calculated to be in excess of 5 billion Norwegian Kroner.

About 75% of the mastitis infections are caused by bacteria. Symptoms vary from animal to animal: some have a high fever and a concomitant reduction of general health state; some appear outwardly healthy, but there are visible changes in the appearance of their milk. Acute clinical mastitis is the most frequently diagnosed disease among milking cattle in Norway.

The Norwegian Agriculture Society is aiming to achieve both a considerable reduction in the frequency of mastitis and also to reduce the quantity of antibiotics used. In May 1996, new guidelines for the treatment of mastitis were given. These guidelines address the need for the administration of antibiotics to be more carefully controlled than has been the case previously. It is a particular aim that treatment should be limited to penicillin-based preparations wherever this is possible. Broad-spectrum antibiotics are to be avoided unless the nature of the infection, and the general health conditions of the diseased animal, definitely merit such treatment. Investigations in Sweden have shown that mastitis caused by infection from the Gram negative bacterium Escerichia coli (E. coli) should not generally be treated with antibiotics at all.

If the aim of a better targeting of antibiotics in therapy, especially mastitis therapy, is to be realised far greater use will need to be made of differential diagnostic tools which can provide a proper on-site guidance to the veterinarian. Only an on-site diagnostic test for mastitis will enable the desired reduction in use of antibiotics since the veterinarian must initiate immediate treatment to maintain a common practical and ethical standard. This cannot be achieved using the conventional diagnostic tools available today.

A proper diagnostic method which can guide the veterinarian will contribute to reduced spread of infectious agents and permit a reduced, but optimal, application of antibiotics. The method currently used to identify and classify bacteria in milk involve cultivations. Such methods take at least one day, and therefore do not satisfy actual diagnostic requirements. A diagnostic test which can identify the cause of mastitis on site is important to enable the best treatment of this disease, and will have wide applicability in many parts of the world.

Similarly, rapid detection and classification of bacteria in liquids like urine, blood, fresh water supplies, solubilised extracts of nutrients etc may lead to more effective therapies less detrimental to the environment. Analyses performed on biological liquids such as blood, urine etc may be associated with administration of antibiotics, analogously to the treatment of mastitis. Infections in the bladder and urine channels in humans and animals are also a considerable source to unnecessary use of broad-spectrum antibiotics. Unnecessary use of antibiotics, and the accompanying negative side effects related to development of bacterial resistance, may be reduced if a rapid method for detection and classification of the bacteria involved was available.

At present, there are a number of products and methods which allow the classification bacteria involved in acute mastitis. These include the so-called Selma-disc (Mybac Vettech, Hagersten, Sweden), a culture medium which after 1 day of incubation with a milk sample permit determination of the presence of the most commonly bacteria involved in mastitis. The method is based on ordinary bacterial culture detection in which a sample is spread out on a disc divided into sections containing different growth media.

The so-called HY-test (Hy Laboratories, Israel) is another test used in relation to mastitis. This test is based on a stick with cultivation media for E.coli and S.aureus, respectively. The test is based on ordinary cultivation technology and requires 1 day of incubation to provide results.

The LIMAST-test (Mybac Vettech, Hagersten, Sweden) is a method based on limulus-hemolysate. The presence of bacterial toxins activates enzymes in the lysate, which in turn have the ability to cleave chromogenic substrates giving rise to a colour that either can be seen with the naked eye or may be read in a spectrophotometer. The test takes 15 minutes to be performed, but involves a number of steps and requires the availability of special equipment. The method is, therefore, less suited for field use. Moreover, the method will only detect Gram negative bacteria. This failing in classification thus leaves an inconclusive result as to whether there is a Gram positive infection present or there is no bacterial infection.

Further methodologies for detection of bacteria are primarily based on cultivation methods, accompanied by microscopy analyses, optionally after staining of the bacteria. Other methods include immunoassays where antibodies specific to a certain bacteria are used. Furthermore, genetic analyses using the so-called PCR technology is increasingly used in bacterial diagnoses. All these methods are, however, time-consuming and additionally require in most cases special equipment that reduce their utility in acute, on-site, situations.

Some rapid immunoassays have been developed for special bacterial diagnosis. For example, Streptococcus A can be analysed in samples taken from the throat within a few minutes using filter-based immunoassays or immunochromatographic sticks whereby an immobilised first antibody binds to antigens from a particular bacterium, after which the bacteria can be visualised by following application of a second antibody conjugated to either a dye, an enzyme producing a visible dye, or to other signal systems.

Different methods have been used for treatment of samples prior to analyses of bacteria. One particular problem has been to develop a method of solubilising milk so that the treated milk can pass a filter which retains bacteria or bacterial fragments, after which the bacteria are stained and the filter is inspected under a microscope. A method introduced in 1980 by Pettipher et al. (Appl. Environ. Microbiol. 39 pp. 423-429) involves mixing a milk sample with a reagent resulting in a final concentration of the components as follows: 0.08-0.4% Triton X-100, ca. 2% W/V Trypsin and chymotrypsin, and 40% of a milk sample. The mixture was subjected to incubation at 50° C. for 10 minutes followed by filtration through a polycarbonate-filter with pore size 0.6 μm. Prior to filtration, the filter was washed with a hot solution of 0.1-0.5% Triton X-100. The filter was subsequently stained with acridine orange which binds to DNA in the bacteria, after which the bacteria were visualised and counted under a microscope. Application of alternative dyes like methylene blue, periodate oxidation followed by basic function staining, toluidine blue and phenol alanine blue was reported to stain additional material which made counting of the bacteria under a microscope more difficult. A prerequisite for DNA-staining methods is also that the samples are treated so that bacteria therein remain sufficiently intact to retain their DNA intracellularly.

Variations of this method are also presented in which:

1) different enzyme mixtures are used and the filter is rinsed with alcohol (Buchrieser and Kaspar (1993) Int. J. Food Microbiol. 20, pp.227-236);

2) citrate-NaOH-buffer at pH 3.0 is used during filtration to improve the process (Ubaldina et al., J. Appl. Bacteriol. (1985) 59, pp. 493-499); and

3) detection utilising antibodies labelled with fluorescent dyes (Tortorello and Gendel (1993) J. Food. Prot. 56, pp. 672-677).

Further optimisation of the method was presented by Fernandez-Astorga et al. in J. Microbiol. Meth. (1995), 24, pp. 111-115. The incubation time was reduced to 1.5 minutes, the filter detergent wash was omitted, and adjustment in the sequence of steps was made in order to improve visualisation of the bacteria for microscopic detection on the filter after staining.

In a further version of this method, the bacteria are visualised under a microscope after incubation with a monoclonal antibody conjugated to alkaline phosphatase. This causes production of an enzymatically derived product, precipitated as a blue stain where the antibodies is bound to the bacteria (Batina et al. J. Appl. Microbiol. (1997) 82, pp. 619-624).

All these methods cited require, however, the use of a microscope.

Filtration methods for the analysis of bacteria in samples not requiring treatment with detergents and enzymes have been described. In U.S. Pat. No. 4,336,337 Wallis and Melnick disclose a method for analysing urine in which the bacteria are treated with a metal chelator and are stained prior to filtration through a filter which has a net positive charge and a pore size allowing retention of the bacteria. Staining with a cationic dye permits visualisation with the naked eye of as few as 10⁵-10⁶ bacteria/mL. A staining method distinguishing Gram+ from Gram− bacteria is also disclosed in this patent. According to this method, the bacteria are stained at basic pH with a cationic substance such as safranin. Then the filter is washed with a solution of an organic acid at a pH of ca. 3.0. Such washing destains Gram+ bacteria, whereas Gram− bacteria remain stained.

Likewise, Longoria et al. in U.S. Pat. No. 5,081,017 presented a method in which bacteria in urine or water are treated with a solution which causes them to be retained in a filter with a net negative surface charge. The filter is then stained, after which the presence of bacteria can be seen visually as staining of the filter.

Similarly, Romero et al. (J. Clin. Microbiol. (1988), 26, pp. 1378-1382) have described a method in which bacterial cultures are subjected to filtration and thereafter classified under a microscope after traditional Gram-staining.

None of these methods have, however arrived at a solution whereby bacteria in milk, other human or animal body liquids, water or solubilised solids extracts can be identified using a filtration method without the use of a microscope. It is, therefore, an object of the present invention to provide a method for detecting the presence on bacteria simply and quickly and without requiring the use of bulky and/or awkward equipment such as microscopes of the like.

Surprisingly we have found that such identification may be effected after adding to a liquid sample (e.g. milk) a reagent having alkaline pH, consisting of detergent(s), salt(s), chelator(s) and optionally organic solvent(s) which are fully or partly soluble in water. The method works in the absence of enzymes and incubation of the sample over prolonged periods of time, and leaves the sample/reagent mixture in a state where it is able to pass a filter which retains bacteria and bacterial fragments.

Furthermore, it has surprisingly been found that in spite of this treatment (which may involve treatment at pH values of up to 13), bacteria are preserved in such a way that they can be retained by a filter with pore size <5.0 μm, preferably <3.0 μm, optimally <1.0 μm, e.g 100 to 1000 nm, for example 200 to 900 nm.

Moreover, it has surprisingly been found that in combination with the described treatments, the bacteria may be stained with different reagents in such a way that bacteria in concentrations of 10⁶/mL can be visualised with the naked eye as a colour on the filter, or optionally as a colour which can be measured by spectrophotometric means.

Visual examination with the naked eye of the colour intensity can even be used to give an estimate of bacterial count.

It has also been surprisingly shown that different staining methods can be utilised whereby to classify the bacteria as Gram negative or Gram positive, and that the different stains correspond to the classification obtained with growth on culture media followed by-staining of the intact bacteria.

An important aspect of the invention is that the method thereof can be performed without a microscope.

In contrast to Wallis et al. (U.S. Pat. No. 4,336,337) we have also surprisingly found that bacteria or bacterial fragments from liquids in general can be retained in certain filters possessing neutral net charge, and thereafter may be stained with a number of dyes with negligible or insignificant background staining of the filter itself.

The present invention thus provides a rapid test suited for the identification and classification of bacteria in liquid samples, or in liquid extracts of solid samples, particularly suitable for the detection of bacteria associated with mastitis in milk. The test is based on a simple principle which offers a rapid performance without requiring the use of advanced equipment. The procedure may be conducted in less than 8 minutes, even in about 4 minutes with moderate training.

The identification/detection is made possible through the presence or absence of colours on the surface of a filter. The presence of a colour indicates a bacterial infection, and the chemical behaviour of the colour serves to distinguish bacteria treatable with penicillin, from those penicillin-insensitive bacteria which thus require alternative treatment. Moreover, it is possible to distinguish Staphylococci from Streptococci by detecting the presence of the enzyme catalase as is known to those skilled in the art; this may be conducted in less than 1 minute.

The most obvious advantage associated with the present invention is the simple and time saving procedure compared to alternative methods, and that the method when applied to diagnosis of mastitis may be used for immediate classification of the bacteria involved, with a concomitant positive consequence for the choice of antibiotics to treat the disease.

An important clinical driving force is to avoid unnecessary pain and suffering for the animals. Furthermore, the method is suited for reduction of unnecessary use of antibiotics, and thus in turn it will contribute to reduction of the spread of antibiotic-resistant bacteria. Furthermore, an early and precise diagnosis may reduce the spread of bacteria associated with mastitis, such as S. aureus, to other animals in the immediate surroundings. An example of a further reason to apply the test is that treatment of animals with unnecessary broad-spectrum antibiotics will lead to interruption in delivery of milk for prolonged periods of time, whilst treatment with penicillin interrupts delivery of milk for only a few days.

Viewed from one aspect, therefore, there is provided a method for analysing for bacteria in a liquid sample comprising the following steps:

a) contacting said sample with a substrate capable of retaining bacteria or bacterial fragments, e.g. filtering said sample through a filter, said filter having a pore size of 5 μm or smaller;

b) staining any bacteria retained on said substrate with at least one solution comprising a first dye, said staining being conducted at a pH at which the dye has an overall positive charge and the bacteria has an overall negative charge; and

c) inspecting, e.g. with the naked eye and/or by spectroscopic analysis, the substrate for the presence of a stain whereby to indicate the presence of bacteria in said sample;

with the proviso that where said liquid sample is other than a milk sample and substrate is a filter then said filter comprises a polysulfone or a derivative of a polysulfone.

The inspection of the substrate, e.g. filter, may be by way of any convenient means such as, for example, with the naked eye and/or with a spectrophotometer and/ or with a microscope. Inspection with the naked eye is preferred.

As discussed further below, particles may be used as the bacteria or bacterial binding substrate; however the discussion below will initially use on the use of filters.

Where said sample is a milk sample it is preferred that, prior to the filtration referred to above, the milk sample is mixed with a reagent having pH of approximately 8 or more; and which contains at least one salt, at least one chelating agent for binding di- or polyvalent cations, at least one detergent and optionally an organic solvent which is fully or partly soluble in water.

Viewed from a further aspect, there is provided a kit for analysing bacteria in a liquid sample comprising the following components:

a) a device comprising a substrate capable of retaining bacteria or bacterial fragments, e.g. a filter having a pore size of 5 μm or smaller, said device optionally comprising a liquid adsorbent material in physical contact with said substrate;

b) a reagent composition having pH of approximately 8 or more and which contains at least one salt, at least one chelating agent for binding di- or polyvalent cations, at least one detergent and optionally an organic solvent which is fully or partly soluble in water;

c) a solution comprising a first dye for staining a bacterium or a bacterial fragment, said dye exhibiting overall net positive charge at a pH of less than about 4;

d) a first washing solution containing an alcohol and a buffer substance, said first washing solution having a pH in the range 2.6-3.5; and optionally

e) a second washing solution comprising at least one detergent.

It is preferred that the filter be a polysulfone or a derivative of a polysulfone.

As mentioned earlier, slight background staining can occur. This may happen with application of certain combinations of reagents; such staining may optionally conveniently be removed by washing with a solution of a detergent, for example 1% Triton X-100. In preferred embodiments of the kits and methods according to the invention, the filters applied for such purposes are made from polysulfone or polysulfone derivatives as for example polysulfone ether. Certain other types of filter materials can also be used, for example polycarbonate. Bacteria are stained with a solution of a dye which in a preferred version of the method and kit is a solution of one or more amine-substituted phenylthiazines, to which group belongs the dye generically known as toluidine blue (for example bisulfite toluidine blue) or amine-substituted phenazines, for example safranine. Additional dyes may also be used. Among such staining means are also colloidal metals covered by oligomeric or polymeric substances containing amine residues. As opposed to Wallis et al. (supra) we have surprisingly found that treatment of stained bacteria with a solution at pH of between 2.7 and 3.5 destains Gram negative bacteria, whereas Gram positive bacteria maintain their colour.

The present invention will now be described in greater detail.

The method and kits of the invention may include a chemical reagent composition for treatment of samples expected to contain bacteria; the chemical reagent allows bacteria in the sample to be retained on a filter after said treatment. Such reagents are generally aqueous liquids or concentrates suited, if necessary after dilution, for treatment of blood and milk and other liquids and contain at least one detergent, one salt, at least one chelator, and optionally one or more organic solvents which are fully or partly soluble in water. Liquids suitable for treating samples not containing lipids or cellular material are typically composed of salts, detergents and chelators.

Many detergents are suitable for inclusion in the chemical reagent used to treat samples. Suitable non-ionic detergents include but are not limited to Thesit, Triton X-100, detergents of the Tween series and the Nonidet series of detergents. Suitable ionic detergents include but are not limited to bile acids, e.g. cholate, deoxycholate, litocholate and chenodeoxycholate and derivatives thereof; Zaponin; and sodium salts of alkyl sulfates, for example sodium dodecylsulfate. Mixtures of the detergents may also be used; in particular a combination of nonionic and ionic detergents is preferred. Relative to the total weight of the reagent composition in ready to use form, the detergents are used in a total concentration of at least 0.05%, preferably 0.2-5%, more preferably 0.5-4.5%, particularly 0.5-2.5%, especially about 1%.

The addition of a chelator capable of binding cations which carry more than one positive charge is preferred and has demonstrated a surprisingly positive effect on the solubilisation of many samples, in particular on milk. The protein fraction in milk is, for example, efficiently solubilised by reagents incorporating a chelate. Useful chelates include, but are not limited to, ethylene diamine tetraacetic acid (EDTA), ethylene glycol-bis(β-aminoethyl ether)-N,N,N′,N′-tetraacetic acid (EGTA), diethylene triamine-N,N,N′,N″,N″-pentaacetic acid (DTPA), 1,2-bis(o-aminophenoxy)-ethane-N,N,N′,N′-tetraacetic acid (BAPTA) or the corresponding tetraacetoxy methyl ester derivative (BAPTA-AM). The chelate or mixture of chelates is/are present in a concentration of 0.02-1 mol/L, and at a pH>8.0. In an especially preferred version of the invention, 0.2 mol/L EDTA is used at pH>10.

Addition of salts too has a positive effect on solubilisation of samples. Salts suitable for this purpose include, but are not limited to, chloride, bromide, nitrate, sulfate, acetate or phosphate salts of monovalent cations such as sodium or potassium. A preferred version of the invention employs 0.05-2.0 mol/L NaCl, typically 0.1-1.0 mol/L.

Addition of organic solvents which are fully or partly soluble in water also have a positive effect on the solubilisation of samples containing lipid or cellular material. Thus, alcohols such as methanol, ethanol, propanol (i.e. propan-1-ol and propan-2-ol), butanol (i.e. butan-1-ol, butan-2-ol, 2-methyl-propan-1-ol and 2-methyl-propan-2-ol), phenols such as phenol itself and aralkanols such as benzyl alcohol, may be used. Further solvents that are suitable include ethylene glycol, acetonitrile, dimethyl sulfoxide (DMSO), dimethyl formamide (DMF), tetrahydrofuran (THF), optionally also present with compounds that are less soluble in water, such as, for example, chloroform, trichloromethane, various alkanes or alkenes, or the halogenated derivatives thereof.

Many of the available filter materials are poorly resistant to some solvents. The preferred solutions will, therefore, vary with the type of filter used. A suitable embodiment of the invention employs 10 volume % acetonitrile in conjunction with filters made from polycarbonate, polysulfone, or polysulfone derivatives.

A reagent mixture particularly suitable for solubilisation of milk in a milk:reagent ratio of 1:5 is: 1% Triton X-100, 0.1 mol/L EDTA pH 12.0, 1.0 mol/L NaCl and 10% acetonitrile. The solubilisation occurs more rapidly at higher temperatures such as, for example when the mixture maintained at between 40° C. and 60° C. for between 30 seconds and 10 minutes, especially 1 to 3 minutes.

The solubilised test sample, (e.g. milk)/reagent mixture is filtrated by applying a positive pressure from above, or a negative pressure from below. Both techniques may be performed using a syringe coupled to a closed filter unit as for example Millex® or Swinnex® (Millipore Corp., USA), or similar equipment obtainable from Pall Gelman Sciences, USA. Negative pressure from below may also be obtained by placing the filter in close contact with a liquid-absorbing, fibrous layer. In such embodiments the concept will be improved by placing the filter under a liquid impermeable layer with a defined aperture exposing part of the membrane. This will limit and define the filter surface, and will moreover result in a concentration of the sample flow. In such embodiments the filter exposed to the sample can preferentially be smaller than the filters used in most of the embodiments described above. A suitable diameter for such purposes is between 0.5-25 mm, although any size may be used. In a preferred version of the method, filter exposures of between 2 and 15 mm are used. It may also be advantageous to place the filter under a water-impermeable material shaped as a tract or as a tube. This will facilitate the throughput of larger quantities of liquid as well as throughput of liquids containing detergents and organic solvents without spoiling. Plastics, casings, supports or containers may form part of the device used for the filtrations. A filter with an underlying, absorbing layer may also be placed in a container connected to an upper tract or tube-shaped component. A closed container will ensure that the user will remain substantially uncontaminated with any of the liquids after these have passed the filter, and the entire unit may be disposed after use.

The filter material used in connection with the present invention must be substantially resistant to the solubilising reagents used. Furthermore, the filter materials must not be stained by the dye(s) used for staining of the bacteria. In a preferred version of the invention, filter materials of polycarbonate, polysulfonate, and derivates of polysulfone (for example polysulfone ether) are used. The filters used have a pore size of 0.2-5.0 μm, typically 0.45-1.2 μm.

Bacteria are known to adsorb a series of dyed substances. Unfortunately, however, it is not uncommon for the samples to contain materials which bind to these same dyes under the same conditions. Consequently unspecific background staining problems can result. Furthermore, several filter materials themselves can absorb such dyes, and it is also common for the filters to absorb proteins and other solubilised materials which in turn absorb the dyes and make distinction from bacterial staining impossible without using a microscope. The above-cited publications all note that successful staining of bacteria on filter materials is dependant on keeping the bacteria intact. It is, therefore, quite surprising that even after the above-mentioned treatment of milk involving a high concentration of detergent, chelator, strongly alkaline pH up to pH 13; and organic solvents, that the bacteria or bacterial fragments are still in a sufficiently intact state that allow them to be stained with selected dyes.

Dyes that may be used for this purpose may, for example, be amine-substituted phenothiazines such as toluidine blue, thionine, and methylene blue; or amine-substituted phenazine derivatives such as safranine, or neutral-red; or amine substituted phenyl-dicyclohexadiene derivatives such as gentian-violet, methyl green and fuchsine-derivatives, or amine-substituted porphyrin derivatives or phthalocyanine derivatives such as alcian blue. Useful dyes are, however, not limited to those listed, and dyes may be used in combinations of 2 or more. In principle, the invention may be effected using any dye exposing a net positive charge at a pH at which the bacteria expose a net negative charge. In classical Gram-staining, either gentian-violet or methyl-violet is added to the sample which thereafter is treated with an iodine solution, followed by washing with ethanol. Gram-positive bacteria from the sample will be stained, whereas the Gram-negative bacteria are initially stained before being destained by the ethanol. Gram-negative bacteria can be counterstained using carbol-fuchsin or safranine.

In another version of this procedure, it has been surprisingly found that some dyes which bind to the bacteria at neutral or weak alkaline pH can be washed off from Gram-negative bacteria when the sample is treated with a solution at pH 2.8-3.5, while the Gram-positive bacteria maintain their stain under the same conditions. In a preferred version of the method, the destaining of Gram-negative bacteria is performed using a mixture of 0.01-0.2 mol/L acetic acid pH 3.0 containing 5-70% ethanol.

In a still further version of the invention, metal colloids are used which conjugate to one or more substances exposing net positive charges at a pH where bacteria expose net negative charges. Metal colloids are intensively coloured and thus may increase by a factor of 10-1000 the sensitivity of the test relative to ordinary dyes. A preferred version of the method employs colloidal gold covered with polymeric or oligomeric compounds carrying primary, secondary or tertiary amine groups. Examples of such compounds are polylysine or oligolysine, chitosan (deacetylated chitine) and basic proteins like histones. Methods to prepare such compounds may be found in the art (Kashio et al. 1992, Histochem. J.24: 419-430; Colloidal Gold (ed: M. A. Hayat), Vol 1-3, Academic Press). The method, however, is not limited to gold colloids since colloids of other metals or salts of metals, as well as colloids of hydrophobic dyes of the kind normally known as disperse dyes, may be used for the same purpose.

Staining according to the procedures of the invention indicates infection by Gram-positive bacteria and prompt treatment with penicillin only. In order to differentiate further between Gram-positive bacteria, a separate aliquot of the sample may be contacted with a solution of hydrogen peroxide. Catalase activity is evidenced through the visible formation of gas bubbles after a few seconds, indicating the presence of Staphylococci, whereas absence of gas bubbles will indicate Streptococci. This may be of additional value in determining subsequent treatment since an optimal regimen with penicillin is different for the two classes of bacteria.

Positive findings of Gram-negative bacteria combined with a serious affection of the animal's general health condition imply that broad spectrum antibiotics such as tetracyclins or sulfa-preparations should be used. If the subject animal's general health condition appears normal, antibiotic treatment should be avoided. A negative result indicate that the disease is not caused by bacteria, but rather have other causes resulting in inflammatory processes. Antibiotic treatment is hence not necessary.

Certain sample materials, such as milk, may contain particular substances which are not readily solubilised by the reagents disclosed herein. In mastitis, the appearance of a typical milk samples containing solid particles, and having a generally slimy appearance are quite common. The direct application of such samples to a filter according to the present invention, may be problematic. However, we have surprisingly found that such samples may successfully be treated by filtration through woolen matrixes or a prefilter with pore size >10 μm, or a combination of those, without significant loss of sensitivity in the subsequently analytical method to detect bacteria through staining. As used herein, the term prefiltration shall be understood as a method to treat samples before the samples are subjected to analyses of bacteria as described elsewhere in this specification.

In a preferred version of the method, the prefilter as well as the woolen matrix are made of hydrophobic materials. The treatment will remove from appropriate sample materials which have a tendency to clog the filters, without significant loss of bacteria. Such prefiltration may be effected to the samples as such, or to samples after admixture with the chemical reagent. In any case, prefiltration, if necessary should be effected before application to the filter.

In one version of this prefiltration method, the prefilter is included in the analytical device in a way that it can be removed after passage of the sample, or the sample treated with said chemical reagent, in order to expose the filter retaining the bacteria or bacterial fragments. In another version of the method, the prefiltration material is provided in a separate device that can be used to prefilter samples before or after admixing with said chemical reagent.

Hydrophobic materials suited for prefiltration of samples as described above include, but are not limited to: polyalkenes such as polyethylene and polypropylene, polyesters, polyvinylchloride, polyurethanes, polyacrylates, polyacrylamides, polysulfones, polyethersulfones, polycarbonates, and nylon. However, it should be understood that any hydrophobic material in general may be used. The material may be shaped as a filter, which has a pore size of >5 μm, preferentially >10 μm. The material may also be used as a woolen matrix which in a preferred embodiment is placed within a container with an inlet and an outlet allowing sample materials to pass through. Optionally, a hydrophobic filter with a pore size >5 μm, preferentially 15-80 μm, is positioned adjacent to the woolen matrix in the container. In one embodiment of the invention, the filtration process may also be performed in a unit, hereinafter filtration unit, which permits lateral liquid movement.

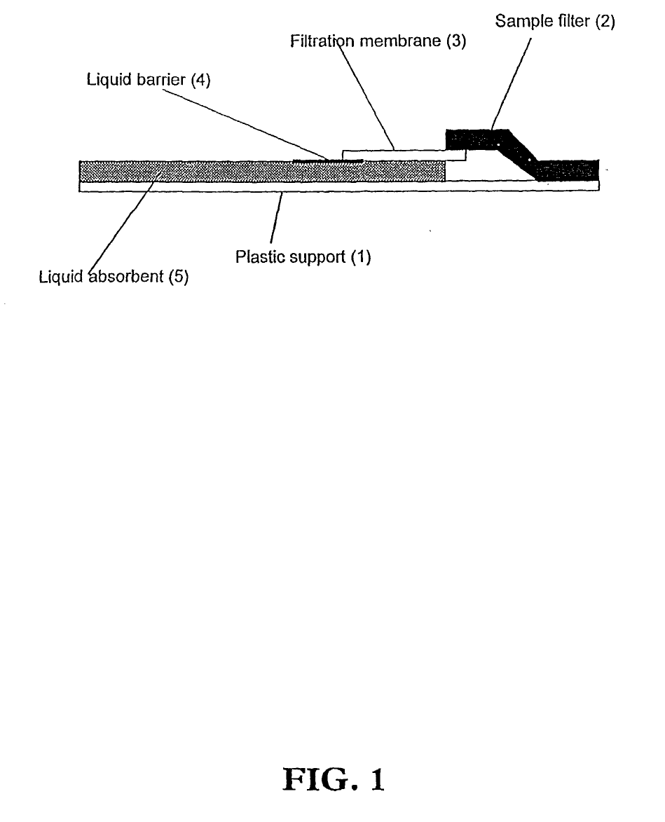

One possible embodiment of such a construction is shown in FIG. 1. Thus, a dipstick, i.e. an elongate strip, may be used in this invention which carries exposed substrate on a surface thereof. The entire unit shown in FIG. 1 is mounted on a plastic support (1). The system may, however, additionally be mounted in a plastic housing which may be useful in some applications is to protect the user and the user's environment against contamination from chemicals and/or microorganisms.

All the layers of the filtration unit are in physical contact with each other, allowing liquids to move by capillary action from the point of application to the other layers.

The sample which, for example, in the case of milk may be solubilised as described elsewhere in this application, is applied to the sample filter (2). Application may be by adding liquid to the sample filter, or by dipping the sample filter part of the filtration unit into the sample.

The sample filter of the filtration unit should preferably be composed of a material that allows bacteria to pass through it, but has a pore size allowing retention of particles which should not pass through the sample filter to other regions of the filtration unit. Appropriate materials for this purpose include those materials used for the prefilter in the prefiltration described above.

These materials should be present as woven or non-woven filter matrices with an average pore size greater than about 5 μm.

The sample filter may be composed of more than one layer; in such cases each layer may or may not have a composition and/or pore size different to the other layers.

Samples applied to the sample filter will move by capillary action to the filter membrane (3) in which any bacteria present are trapped. The filter membrane should at least have some overlap with the liquid absorbent (5).

Where overlap is great, the filter membrane will act substantially as a vertical filter, whereas less overlap will render the filter membrane as a laterally working filter. The filter membrane is preferably made from either a polycarbonate, a polysulfone or derivatives thereof. The pore size is preferably in the range 0.2-5.0 μm, especially preferably 0.45-2.0 μm.

The sample liquid is allowed to pass through the sample filter at least until wetting is visible on the remote side of the liquid absorbent (5). The filter membrane should at least partly overlap with the liquid absorbent (5). Once the liquid is visible on the remote side of the liquid absorbent (5), any bacteria present on the filter membrane may be visualised by application thereto of a dyed substance as described elsewhere in this specification. The dye is preferably applied directly to the filter membrane and the liquid allowed to migrate into the liquid absorbent.

This step may optionally be followed by application of one or more washing solutions as described elsewhere in this specification, including those solutions which destain Gram negative bacteria. The final result, as in other embodiments of the invention is read as the presence or the absence of colour on the filter membrane. The amount of colour, judged as a combination of colour intensity and the width of the coloured zone, may also be used to judge quickly the quantity of bacteria present.

The liquid absorbent (5) should have sufficient capacity to absorb the total volume of liquids applied to the filtration unit.

In a preferred embodiment of the filtration unit of the invention, is embedded in a plastic housing with apertures exposing the sample filter and the filter membrane. Application of sample is thus made through the sample filter aperture, and the further liquids are applied through the filter membrane aperture, through which the final result may also visualised.

In a yet further aspect of the invention, beads or particles may be firstly used to bind the bacteria in the liquid samples, followed by bacterial staining. Preferably, the staining may be visualised by concentrating the beads by centrifugation or filtration. Magnetic beads may be used in which case the colour from stained bacteria attached to the beads may be visualised after attracting the beads to the inner surface of a test tube or the wall of any chamber by means of a magnet.

An advantage of this aspect of the invention is that small amounts of bacteria contained in rather large volumes of liquid may be concentrated on the surface of beads, and the beads may further be concentrated as described above allowing a direct visualisation of their presence and Gram stain characteristics. It should be understood that other staining methods for bacteria may be applied, as for example staining of bacterial DNA with acridine orange.

In order that bacteria may be attached to the beads, the surface of the beads should contain structures that permit binding to the bacterial surface. Tosyl-activated beads like the magnetic Dynabeads® M-280 (Dynal AS, Oslo, Norway), may be used for this purpose. When mixed with a medium containing bacteria, amino groups on the bacterial surface will react with the tosyl groups on the beads whereby to couple the bacteria to the beads. The beads may then be filtered or concentrated on the wall of, for example, a test tube by means of a magnet then resuspended in a liquid containing a dye such as methyl violet, malachite green, toluidine blue, a fuchsin or any other staining material as described elsewhere in this application.

In an alternative embodiment of this variant, a milder type of binding of the bacteria is used to avoid side reactions associated with unspecific binding from other compounds in the medium. One such convenient method is to coat beads with poly-lysine. Such coating may be performed onto tosyl-activated beads that readily bind poly-lysine, and which leaves a plurality of amino groups on the poly-lysine free if the concentration of poly-lysine relative to the bead surface is sufficient. The poly-lysine coated beads readily attract bacteria that subsequently may be stained on the surface of the beads.

In general, any method whereby chemical groups carrying positive charges are introduced to the surface of the beads, may result in a bead attracting bacteria to its surface. The beads may be magnetic, like the Dynabeads® mentioned above, or any bead of a reasonable size modified to carry positive charges. Such beads may be ordinary polystyrene latex, or beads made of any polymeric material. Also, certain types of beads carry negative charges in themselves and may be used without further modification.

Non-magnetic particles may be concentrated or collected by centrifugation, and a sequence of washing, staining and destaining steps may readily be performed by repeated centrifugations, removal of the supernatant, and resuspension in a new liquid medium.

Alternatively or additionally, beads carrying bacteria may also conveniently be collected onto a membrane with a pore size which is smaller than the diameter of the beads. The washing, staining and destaining steps may be performed simply by passing the various liquid media through the filter. Filters suitable for this purpose are described elsewhere in this application. Those made of polycarbonate, from a polysulfone, a polyethersulfone, or derivatives of these polymers are preferred.

The invention will now be illustrated by way of the following non-limiting examples.

### EXAMPLES

### Example 1

1 mL of a milk sample was mixed with 4 mL of a solution containing 0.1 mol/L EDTA adjusted to pH 12 with a stock solution of 5 mol/L NaOH, 1.0 mol/L NaCl, 0.5% Trition X-100, and 10% acetonitrile. Heating under hot tap water (ca. 50° C.) for 2-3 minutes reduced the time to obtain solubilisation. The mixture was subjected to filtration through a polypropylene-filter with average pore size of about 45 μm, and thereafter transferred to a syringe which was coupled to a Millex® filtration unit (Millipore Inc.). The filter unit was equipped with a Supor-filter (polysulfone filter from Pall Gelman Inc.) having a diameter of 25 mm. The solution was slowly passed through the filter with a positive pressure from above, using the syringe, after which the filter was washed with 1 mL 1% Triton X-100. 1 mL of a solution of safranin-o (2.5 mg/L in distilled water) was passed through the filter followed by a washing with 1% Triton X-100. The presence of bacteria in amounts of >5×10⁶/mL in the sample was seen as a reddish colour on the filter, whereas samples containing less than the above-mentioned concentration of bacteria appeared as a white, unstained filter. The addition of 1 mL of a solution of 10% ethanol in 0.1 mol/L acetic acid, pH 2.9, destained a previously filter stained due to the presence of Gram negative bacteria. When Gram positive bacteria were present, the filter was not destained.

In order to document the method, aliquots of normal milk were added cultures of Staphylococcus aureus (Gram positive) and Eschericia coli (Gram negative) in increasing concentrations from 1×10⁵ to 5×10⁸ bacteria per mL. Reddish staining of the filters was visible from a concentration of 5×10⁶ bacteria/mL for both stems of bacteria. only the filters with E.coli were destained upon washing with 10% ethanol/0.1 mol/L acetic acid (pH 2.9), while the filters with S.aureus maintained their colour under the same conditions,

### Example 2

The method in Example 1 was repeated exchanging safranin-o with toluidine blue (4.6 mg/l in distilled water). Application of Staphylococcus aureus in an amount of >5×10⁶/mL was shown as a blue stain on the filter. This Gram positive bacteria was not destained upon washing with 10% ethanol/0.1 mol/L acetic acid (pH 2.9), as described in Example 1. The Gram-negative bacteria Eschericia coli in an amount of >1×10⁶/mL was also appearing as a blue stain on the filter, but in this case the colour was removed by washing with 10% ethanol/0.1 mol/L acetic acid (pH 2.9).

### Example 3

The methods of example 1 and 2 were repeated with cow milk samples diagnosed with acute mastitis. To establish a reference, the samples were cultivated on suited media after which the bacterial species and number were determined. Furthermore, the number of somatic cells present was determined using the Schalm test in which the result is given in a scale from 0 (little) to 5 (much). The same milk samples were treated as described in Example 1 and 2 and the results of this test were compared to the results from the reference cultivation method.

The results were as follows:

Sample Schalm test/_Cultivation methodology_New rapid test

| no. | CMT(1) | Bacterial species | Gram char. | Bacterial mL | Colour intensity(2) | Gram. char. (3) | Remarks |
|---|---|---|---|---|---|---|---|
| 1 | 2 | None | 0 | 0 | 0 | 0 | |
| 2 | 4 | Bacillus sp. | G+ | >10⁶ | 4 | G+ | |
| 3 | 4 | S.aureus+ KNS | G+ | 2-2.5 × 10⁴ | 3 | G+ | |
| 4 | 4 | S.aureus | G+ | 1.5-2 × 10⁴ | 2 | G+ | |
| 5 | 5 | None | 0 | 0 | 0 | 0 | |
| 6 | 2 | Coliform | G− | 1-1.5 ×10⁴ | 3 | G− | |
| 7 | 4 | α-streptococci | G+ | 1-1.5 ×10⁴ | 1 | G+ | |
| 8 | 4 | KNS | G+ | >5 × 10⁵ | 5 | G+ | |
| 9 | 1 | None | 0 | 0 | 0 | 0 | |
| 10 | 5 | KNS | G+ | >5 × 10⁴ | 1 | G+ | weak staining |
| 11 | 4 | None | 0 | 0 | 0 | 0 | |
| 12 | 4 | unspecifc | — | not applicable | 3 | G− | probably dominated |
| | | bacterial mixture | | | | | by G− bacteria |
| 13 | 1 | None | 0 | 0 | 0 | 0 | |
| 14 | 5 | α-streptococci | G+ | 2-2.5 × 10⁴ | 2 | G+ | |
| 15 | 5 | S.aureus | G+ | 35 | — | — | not filterable 4) |
| 16 | 5 | None | 0 | 0 | — | — | not filterable 4) |
| 17 | 5 | S.aureus | G+ | 1.5-2 × 10⁴ | 2 | G+ | difficult to |
| | | | | | | | filter 4) |
| 18 | 5 | KNS | G+ | 5 × 10³ | — | — | not filterable 4) |
| 19 | 5 | Coliform | G− | 2 × 10³ | 2 | G− | |
| 20 | 5 | bacterial mixture | — | 1-1.5 × 10⁴ | 2 | G− | antagelig G− dominert |

1) Schalm-test/CMT: test for presence of somatic cells in scale 1-5, where 5 represents the highest cell count

2) Staining intensity evaluated in a scale 1-5 where is the strongest colour, 0 is no visible staining

3) The rapid method characterises into G+ or G− dependent on the characteristics of the staining

4) The samples are all from the same animal over a certain period of time, measured after storage in refrigerator. Experiences with testing shows that cool storage may reduce filterability

From this experiment it may be concluded that the method for detection of bacteria in milk correspond to the results obtained with cultivation down to a level of 10³ living bacteria/mL. This is 2-3 orders of magnitude better than obtained when bacteria were added to fresh, normal milk where the sensitivity was 5×10⁶ for Gram positive bacteria S.aureus) and 1×10⁶ for Gram negative (E.coli). The increased sensitivity is probably caused by detection of also the dead bacteria in the rapid method, which then is not represented in the cultivation studies.

### Example 4

Cultures of Staphylococcus aureus and Eschericia coli were grown in LB (Luria Bertani) medium overnight, and normal milk was mixed with each of the cultures in the ratio 1:2. In addition, a control not containing bacterial cultures was made. An aliquot of 1 mL was collected from each of the mixtures and mixed with 4 mL of a solution containing 0.2 mol/L EDTA adjusted to pH 13 with a solution of NaOH, 0.2 mol/L NaCl, 1% Trition X-100, and 10% acetonitrile. The final mixtures were then transferred to syringes that were coupled to Millex® filtration units containing polycarbonate filters (Isopor®, Millipore Inc.) with pore size 0.4-0.8 μm and a diameter of 25 mm. The mixtures were passed through the filters using the syringes, applying a positive pressure from above. Next, each of the filters were washed with 1 mL of 0.1% Triton X-100. The filters were finally stained as follows: 1 mL of a solution containing 0.5g/L methyl violet 6B and 0.1% Triton X-100 was passed through each of the filters. The filters were washed with 1 mL 0.1% Triton X-100. The filters used for filtration of samples containing bacterial cultures were stained blue. Next, 1 mL of a solution containing 0.1 g/L iodine—0.2g/L potassium iodide—0.1% Triton X-100 was passed through the filters using the syringes. The filters were washed with 1 mL 95% ethanol and washed with 1 mL 1% Triton X-100. Filters used with samples containing the Gram positive S.aureus bacteria were stained dark blue, whereas the filters used with samples containing the Gram negative E.coli or samples not containing bacteria were stained slightly pink. The filters were furthermore added 1 mL of a solution containing 0.1 g/L basic fuchsin—0.04g/L phenol—0.05% ethanol—0.1% Triton X-100, and finally washed with 1 mL 0.1% Triton X-100.

Filters used with samples containing the Gram positive bacteria S.aureus maintained their dark blue staining, whereas filters used with filters containing the Gram negative E.coli were stained red. Filters used with samples without bacteria appeared white and non-stained, and could clearly be distinguished from filters used with samples containing bacteria.

### Example 5

The methods of examples 1 and 2 were applied to filter units with an absorbent layer of cellulose paper underneath the filters. The filters as well as the absorbent layers were assembled in a plastic device with a circular aperture exposing part of the filter. The results were similar to those obtained in Examples 1 and 2 were the liquids were passed through the filters by means of a positive pressure from a syringe.

### Example 6

Urine samples were mixed with the medium described in Example 1, except that acetonitrile was omitted, then filtered, treated and stained as described in Examples 2. The results are listed in the table below. It can be seen that the method correctly permits bacterial classification and estimation of the intensity of the infection.

| | | | | Result/Rapid method | |
|---|---|---|---|---|---|
| Animal | Cultivation studies. | | | Colour | |
| species | Bact. species | Count/mL | Clinical Judgement | intensity | Gram char. |
| Cat | S. intermedius | 2-3 × 10³ | no infection | 0 | — |
| | | | (contamination) | | |
| Dog | B. faecalis | 6 × 10⁶ | G+ infection | 2 | G+ |
| Horse | no growth | | no infection | 0 | — |
| Dog | E. coli | 1 × 10⁶ | G− infection | 3 | G− |
| Cattle | A. pyogenes | >10,000 | G+ infection | 4 | G+ |
| Cat | Klebsiella sp. | 1 × 10⁴ | G− infection | 3 | G− |
| | and Proteus sp | | | | |
| Dog | Proteus sp. | 4 × 10⁶ | G− infection | 4 | G− Cat |
| | no growth | | no infection | 0 | — |
| Dog | no growth | | no infection | 0 | — |
| Dog | E. coli | 3 × 10⁷ | G− infection | 4 | G− Cattle |
| | E. coli | 1.3 × 10⁵ | G− infection | 1 | G− |
| Horse | no growth | | no infection | 0 | — |
| Dog | Staph. sp. | 8 × 10² | G+ infection | 3 | G+ |
| Horse | S. aureus | 1.5 × 10⁷ | G+ infection | 4 | G+ |
| Dog | E. coli | 3 × 106 | G− infection | 4 | G− Dog |
| | Kiebsiella sp. | >106 | G− infection | 1 | G− |
| Dog | Kiebsiella sp. | 2.5 × 104 | G− infection | 3 | G− |
| Pig | E. coli + proteus | 108 | G− infection | 4 | G− Dog |
| | S. aureus | 105 | G+ infection | 2 | G+ |

### Example 7

Cultures of a series of bacterial species with proven identities were grown in liquid media and added milk in the ratio medium:milk=1:5 to a finale number of 10⁷-5×10⁸ bacteria/mL. The final dilutions were tested with the rapid filtration system according to the method in Examples 1 and stained as in Example 2. The results were as follows:

| | | Result with rapid method | | |
|---|---|---|---|---|
| | Gram | Stain | Stain | |
| Bacterial species | charact. | step 1 | step 2 | Conclusion |
| Staphylococcus aureus | G+ cocci | + | + | G+ |
| Eschericia coli | G− rods | + | − | G− |
| Listeria monocytogenes | G+ rods | + | + | G+ |
| Streptococcus agalacrtiae | G+ cocci | + | + | G+ |
| Enterococcus faecalis | G+ cocci | + | + | G+ |
| Pseudomonas aeruginosa | G− rods | + | − | G− |
| Bacillus sp. | G+ rods | + | + | G+ |
| Streptococcus dysgalactiae | G+ cocci | + | + | G+ |
| Klebsiella sp. | G− rods | + | − | G− |
| Staphylococcus epidermis | G+ cocci | + | + | G+ |
| Staphylococcus haemolyticus | G+ cocci | + | + | G+ |
| Arcaobacterium pyogenes | G+ rods | + | + | G+ |

The results show that all bacterial species were stained with the rapid method, and furthermore that the rapid method classified all bacterial species correctly as either Gram positive or Gram negative.

### Example 8

Some samples from cows diagnosed with mastitis have a slimy or particulate appearance that makes them less suited for direct application to the filtration procedure. Such samples will frequently clog the filter and disturb the further analytical process. Such samples constitute 5-10% of the field samples from mastitis cows. Prefiltration of such samples restore their ability to be analysed in the system described in the previous examples.

Prefiltration was performed by construction of a device, using a plastic syringe with a polystyrene sinter positioned in the bottom, covering the outlet. On the top of this is positioned a polypropylene filter with an average pore size diameter of 80 μm, and at the top of this filter a 2-10 mm layer of polystyrene wadding.

Fifteen milk samples with a slimy appearance were selected for analyses directly according to the method described in Example 2, or subjected to such analysis after passage through the prefiltration device. The results for the individual samples are listed in the table. It can be seen that prefiltration improves applicability of the further method to almost 100%, without significant loss of analytical sensitivity.

| | | Amount of sample | | | | |
|---|---|---|---|---|---|---|
| | | passing filter | | Stain intensity | | |
| Bacterial | Bact. | Without | After | Without | With | Gram |
| species | count/mL | prefiltration | prefiltration | prefiltration | prefiltration | charact. |
| E. coli | 10⁵ | 100% | 100% | 2 | 3 | G− |
| Streptococcus | 10⁶ | 30% | 100% | 2 | 2 | G+ |
| S. aureus | 5 × 10⁴ | 100% | 100% | 1 | 1 | G+ |
| E.coli | 10⁵ | 25% | 100% | 2 | 3 | G− |
| KNS + Strept. | 10⁵ | 100% | 100% | 2 | 1 | G+ |
| Streptococcus | 10⁵ | 75% | 100% | 2 | 1 | G+ |
| E.coli | 10⁶ | 100% | 100% | 1 | 3 | G− |
| No bacteria | — | 50% | 100% | 0 | 0 | — |
| Klebsiella sp. | 2 × 10⁴ | 90% | 100% | 2 | 3 | G− |
| E. coli + unspec. | 5 × 10³ | 80% | 100% | 3 | 3 | G− |
| No bacteria | — | 100% | 100% | — | — | — |
| S.aureus | 3 × 10³ | 0% | 100% | 2 | 2 | G+ |
| S.aureus | 5 × 10⁴ | 100% | 100% | 2 | 2 | G+ |
| Streptococcus | 10⁵ | 0% | 100% | 2 | 1 | G+ |
| S.aureus | 10⁵ | 50% | 100% | 1 | 3 | G+ |

### Example 9

A 5 m wide filtration unit was made by gluing a sample filter of 10 mm length to a plastic support. The sample filter was a Sefar Propyltex 30-100 μm pore size material. The sample filter overlapped a filter membrane (Gelman or Pall polysulfone with a pore size of 0.8 μm) by about 2 mm. The filter membrane had a length of 10 mm and was in turn layered over a liquid absorbent (Whatman grade 17 CHR cellulose) width 8 mm. The filter membrane was held in place in relation to the other layers by wrapping a tape partly over the filter membrane, leaving about 5 mm of this membrane exposed to aid further liquid additions and visual inspection.

Milk to which had been added cultures of either Staphylococcus aureus or Escerichia coli (to 1×10⁷ bacterial/mL) was used to-investigate the performance of the filtration unit. 100 μl of each milk sample was added to 100 μl of 0.1 mol/l EDTA adjusted to pH 12.0 with a solution of NaOH/1.0 mol/l NaCl/0.5% Triton X-100. This treatment facilitated the liquid flow and improved the overall test performance. Milk not treated in this way could also be successfully taken up by the filtration units but the method works preferably with treated milk. Filtration units were dipped with the sample filter end about 5 mm into milk samples added bacteria, and milk without bacteria as a control. The liquid was allowed to migrate along the filtration unit until the liquid front reached about 2 mm over the filter membrane zone into the liquid absorbent. The filtration units were removed from the milk samples and placed horizontally. One drop of 1% Triton X-100 was dropped onto the filter membrane and allowed to suck in. One drop of 0.133 mmol/L of toluidine blue was dropped onto the filter membrane and the liquid was allowed to suck in. One drop of 1% Triton X-100 was added.

The filtration units dipped in milk-containing bacteria were stained blue on the filter membranes on a distance of about 2 mm beyond the sample filter, whereas no stain was visible in the filtration units dipped in control milk.

Finally, one drop of ethanol adjusted to pH 2.9 with acetic acid was dropped on the filter membrane. The filtration unit containing E.coli was completely decolourised, whereas the filtration unit containing S. aureus maintained the blue stain.

### Example 10

Filtration units as described in Example 9 were made, but exchanging the sample filter with different materials. Filtration units were thus made with sample filters made of Porex Polypropylene 70-130 μm, or Porex Polyethylene 40-100 μm. The filtration units were tested as described in Example 9 with a similar result.

### Example 11

In a further experiment, the filtration unit described in Example 9 was modified by wrapping a layer of a Gelman Polypropylene filter with a pore size in the range of 30-70 μm around the sample membrane. The polypropylene filter was able to prevent larger particles to enter the sample membrane and thus also prevented the sample filter from being clogged when mastitic milk samples were tested with the filtration unit.

### Example 12

Filtration units were made as described in Examples 9, 10 and 11, using a 8 cm long 0.015 super white polystyrene with GL-187 acrylic pressure sensitive adhesive laminated to one side, and supported with A74 LB Poly Coated Silicone release liner (Gelman).

A Whatman 6 cm long and 0.8 cm wide development material Grade CD427-07 glued onto the right end of the support and constituted the liquid absorbent layer. A 1.5 cm long polysulfone membrane (Gelman) with a pore diameter of-0.8 μm, was positioned over the left side of the absorbent layer. A 3.5 cm long polypropylene prefilter with a pore diameter of 10 μm (Gelman) was positioned in the left end of the filtration unit (as illustrated in FIG. 1) so that it partly overlapped about 0.5 cm over the polysulfone membrane. The polypropylene prefilter was also glued to the plastic support whereas the polysulfone-membrane was kept in position by wrapping a tape around its right part.

Staphylococcus aureus ATCC 25923 and Escerichia coli were cultured overnight and added to milk aliquots to finally concentrations of 10⁶-5×10⁸ bacteria/ml. 100 μl of each milk sample was added to 100 μl of 0.1 mol/l EDTA adjusted to pH 12.0 with a solution of NaOH/1.0 mol/l NaCl/0.5% Triton X-100. This treatment facilitated the liquid flow and improved the overall test performance. Further improvement was achieved when the mixtures were heated under hot tap water (ca. 40-50° C.) for a about two minutes.

A filtration unit as described above was dipped into each of the samples and the liquid was allowed to be absorbed. 100 μl 1% Triton X-100 was applied to the polysulfone membrane, followed by 100 μl of a solution of 133 μmol/l toluidine blue, and 100 μl 1% Triton X-100.

The presence of bacteria could be seen as a blue colour at the polysulfone membrane at concentrations as low as 10⁶ bacteria/ml.

Application of 100 μl of 10% ethanol adjusted to pH 2.9 with acetic acid destained the colour on filtration units containing E.coli whereas filtration units containing S.aureus maintained their colour, thus distinguishing between Gram positive and Gram negative bacteria.

### Example 13

One colony from fresh culture of each of different bacterial species was inoculated into Luria Bertani (LB) or Heart Infusion (HI) broth (Difco, Detroit, Mich., USA) and incubated at 37° C. overnight. 50 μl (10 mg/L) tosyl-activated monodisperse magnetic particles (Dynabeads M280, Dynal, Oslo, Norway) was added to 1.25 ml bacterial culture in a 1.5 ml microtube and incubated at 200C for 10 minutes with end over end mixing. Uninoculated LB- or HI media were used as negative controls. After incubation, the beads were washed once with 1 ml distilled water. The beads were resuspended in 200 μl 500 mg/l methyl violet (Sigma, Mo, USA) and incubated at 200C for 1 minute. After incubation, the beads were washed with 1 ml distilled water. The beads were resuspended in 200 μl 2500 mg/l iodine (Sigma, Mo, USA) and incubated at room temperature for 30 seconds and subsequently washed with 200 μl 96% ethanol followed by washing with distilled water. Concentrated beads were resuspended in 100 μl 1000 mg/l carbolfuchsin (Sigma, Mo, USA) and incubated at 20° C. for 30 seconds and then washed in 1 ml distilled water. Washing steps consisted of concentrating the beads for 30 seconds in a Magnetic Particle Concentrator for microtubes (MPC®-M, Dynal, Oslo, Norway) followed by aspiration of the supernatant. Finally the beads were resuspended in 100 μl distilled water and then concentrated of the wall of the microtube by using MPC for microtubes. Bacterial cultures that retained the violet colour on the bead surface were termed Gram positive, and those staining red from the carbolfuchsin counterstain were termed Gram negative. Negative controls appeared colourless.

For microscopic examination, a 10 μl magnetic beads-bacteria suspension was transferred to a slide, covered by a glass cover and added one drop immersion oil before viewing (x100).

The technique was applied to a range of bacterial suspensions. The method correctly identified the Gram stain of overnight cultured suspensions of the following clinically relevant bacteria: Staphylococcus aureus ATCC 25923, Staphylococcus intermedius, Streptococcus dysgalactiae, Streptococcus zooepidemicus, Escerichia coli, Pasteurella multocidia, Pseudomonas aeroginosa, Bacillus subtilis, and Proteus mirabilis.

### Example 14

Tosyl-activated monodisperse magnetic particles (Dynabeads M280, Dynal, Oslo, Norway) was coated with poly-lysine as follows. A suspension containing 5 mg M280 beads was washed with 0.1 M Na-phosphate buffer (pH 7.4)-as recommended by the manufacturer. The beads were finally resuspended in the same buffer to obtain a concentration of M280 beads of 5 mg/ml. 200 μg poly-Lysin (Sigma. St.Louis, USA) dissolved in 1 ml of the suspension buffer was added and the solution was incubated at 37° C. with end-over-end mixing for 24 hours. The beads were washed twice with a solution of 0.01 M Na-phosphate/0.15M NaCl/0.1% bovine serum albumin (BSA) and finally resuspended in 0.2 M Tris-HCl buffer (pH 8.5)/0.1% BSA and incubated at 37° C. with end-over-end mixing for another 24 h.

The procedure in example 1 was repeated by addition of 100 μl bead suspensions to 1.25 ml media containing bacteria, or control media without bacteria, and incubated by end-over end mixing for 30 minutes, followed by washing and staining as above. The final staining of the beads correctly identified the Gram-characteristics of the various bacterial strain.

### Example 15

The procedure in example 14 was repeated, but after incubation of the beads with bacterial cultures, the suspension was filtered through a Supor polysulfone filter (Pall Gelman Inc.) leaving the beads with bacteria attached on the filter surface. The filter was mounted in a Millex Filtration unit (Millipore Inc.) and the liquid was applied with a syringe. The staining sequence described in example 1 was repeated by application of the various liquids to the filter unit, and incubation at the desired time intervals before each liquid was finally passed through the filter.

The staining appeared on the filter according to the Gram-characteristics of the bacteria, and identified the strains listed in Example 1 correctly

### Example 16

The procedure in Example 15 was repeated up to the start of the staining procedure. The staining was performed by passing though the filter 1 ml of a solution containing 4.6 mg/l toluidine blue. The filter was washed with 1 mL 1% Triton X-100. All filters containing beads incubated with bacteria were stained blue, whereas the control filters has no sign of blue staining.

The filters were further washed by passing through 1 ml 0.1 M Na-acetate buffer pH 2.9/10% ethanol. The filters containing the Gram positive bacteria were unchanged whereas the filters containing Gram negative bacteria were destained.

1. A method for analysing for bacteria in a liquid sample comprising the following steps:
a) contacting said sample with a substrate capable of retaining bacteria or bacterial fragments;
b) staining any bacteria retained on said substrate with at least one solution comprising a first dye, said staining being conducted at a pH at which the dye has an overall positive charge and the bacteria has an overall negative charge; and
c) inspecting the substrate for the presence of a stain whereby to indicate the presence of bacteria in said sample;
with the proviso that where said liquid sample is other than a milk sample and substrate is a filter then said filter comprises a polysulfone or a derivative of a polysulfone.

2. A method according to claim 1 wherein said liquid is a human or animal body liquid, a liquid nutrient extract or water.

3. A method according to claim 1 or claim 2 wherein said liquid is milk.

4. A method according to claim 3 wherein, prior to step a) said milk sample is contacted with a reagent with pH higher than about 8.0, said reagent containing salts, at least one chelator binding di- or polyvalent cations, at least one detergent and optionally an organic solvent which is fully or partly soluble in water.

5. A method according to any preceding claim wherein said substrate is a filter having a pore size of less than or equal to about 5 μm; or is a bead or a particle having a surface containing structures whereby to permit the binding of bacteria; or is exposed and is carried on the surface of a dipstick.

6. A method according to claim 5 wherein said filter comprises a polysulfone, a polysulfone derivative or a polycarbonate.

7. A method according to claim 5 wherein the surface of said bead or a particle comprises tosyl groups.

8. A method according to either claim 5 or claim 7 wherein said bead or said particle is magnetic.

9. A method according to any preceding claim wherein subsequent to step c) the following steps are conducted:
i) washing said substrate with a solution containing an alcohol and optionally a buffer substance suited to obtain a pH in the range 2.6-3.5;
ii) classifying the bacteria retained on said substrate as Gram positive if the colour on the substrate is maintained as Gram negative if the substrate is destained; and optionally, where the substrate is destained
iii) contacting said substrate with a second dye suited for staining Gram negative bacteria; and
iv) classifying the bacteria retained on said substrate as Gram negative if the colour on the substrate is stained with said second dye.

10. A method according to any preceding claim wherein said first and second dyes are selected independently from the group comprising amine-substituted derivatives of a phenothiazine, phenazine, phenyl-dicyclohexadiene, hemocyanin, or colloidal metals covered by amine-substituted oligomers or polymers.

11. A method according to any preceding claim wherein prior to application to said substrate, said sample is prefiltered through a porous matrix comprising a hydrophobic woolen material, or a prefilter made of a hydrophobic material, or a combination of a woolen hydrophobic material and a hydrophobic filter, said prefilter having a pore size >5 μm.

12. A kit for analysing bacteria in a liquid sample comprising the following components:
a) a device comprising a substrate capable of retaining bacteria or bacterial fragments, said device optionally comprising a liquid adsorbent material in physical contact with said substrate;
b) a reagent composition having pH of approximately 8 or more and which contains at least one salt, at least one chelating agent for binding di- or polyvalent cations, at least one detergent and optionally an organic solvent which is fully or partly soluble in water;
c) a solution comprising a first dye for staining a bacterium or a bacterial fragment, said dye exhibiting overall net positive charge at a pH of less than about 4;
d) a first washing solution containing an alcohol and a buffer substance, said first washing solution having a pH in the range 2.6-3.5; and optionally
e) a second washing solution comprising at least one detergent.

13. A kit according to claim 13 wherein said substrate is a filter having a pore size of less than or equal to about 5 μm; or is a bead or a particle having a surface containing structures whereby to permit the binding of bacteria; or is exposed and is carried on the surface of a dipstick.

14. A kit according to claim 13 wherein said substrate is a filter comprised of a polysulfone, a polysulfone derivative or a polycarbonate.

15. A kit according to claim 13 wherein said substrate is a bead or a particle and said surface comprises tosyl groups.

16. A kit according to either claim 13 or claim 15 wherein said bead or said particle is magnetic.

17. A kit according to any of claims 13 to 16, comprising additional means to remove insoluble material from the sample prior to contacting said substrate, characterised by being a device containing a prefilter with poresize >5 μm, or containing a woolen material, or containing a combination of a prefilter and a woolen material, said prefilter and/or woolen material being made from a hydrophobic material.
